(19) United States
(12) Patent Application Publication
(10) Pub. No.: US 2007/0144534 A1
(43) Pub. Date: Jun. 28, 2007

(54) System to prevent airway obstruction

(76) Inventors: Carlos Mery, Mountain View, CA (US); Bilal Shafi, Palo Alto, CA (US); Gary Binyamin, Palo Alto, CA (US); Jessica Anne Connor, Palo Alto, CA (US); John White, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/607,669

(22) Filed: Nov. 30, 2006

Related U.S. Application Data
(60) Provisional application No. 60/740,854, filed on Nov. 30, 2005.

(51) Int. Cl. A61F 5/56

(52) U.S. Cl. 128/848

(57) ABSTRACT

Devices and methods are provided for the treatment of obstructive sleep apnea. An implantable device is inserted into the tongue. The implantable device is a flexible elongated structure, which may be a curved or jointed filament; of a sufficient length and diameter to provide support, when implanted, sufficient to restrain obstructive movement of the tongue during sleep.

### TECHNICAL FIELD

This invention relates generally to the treatment of patients with obstructive sleep apnea, and more specifically to an improved minimally invasive, reversible system to prevent airway obstruction in a patient with obstructive sleep apnea.

### BACKGROUND OF THE INVENTION

Over 15 million Americans suffer from obstructive sleep apnea (OSA), an under appreciated disease that in its most severe form can have dire health consequences and lead to premature death. Obstructive sleep apnea is characterized by instability of the upper airway occurring during sleep and leads to frequent episodes of breathing cessation (apnea) or decreased airflow (hypopnea). During these episodes, the patient has a brief arousal from sleep that allows restoration of airway patency and resumption of breathing. The segmentation of sleep derived from these episodes of “nocturnal asphyxia”, which can occur as much as 400-500 times per night, leads to excessive daytime somnolence. Hypersomnolence can become disabling and dangerous; studies show that patients with OSA have two to seven times more motor vehicle accidents than people without OSA. In addition, these episodes can also cause intellectual impairment, memory loss, personality disturbances, impotence, arrhythmias, hypertension, heart attacks, stroke, and premature death.

When a person is awake, the muscle tone of the pharynx acts to maintain the airway open against the negative pressure of inhalation. Patients with OSA appear to have more redundant and “floppy” tissue in the pharynx leading to a higher likelihood of airway collapse during sleep, when the tongue and other muscles lose their tone. The airway structures that are known to be involved in the pathophysiology of the obstruction in OSA are the tongue, the soft palate, and the lateral walls of the pharynx.

Studies have shown that the tongue is one of the major contributors to obstruction in 65% of the patients. Although the soft palate has been found to be involved in 85% of the patients, the tongue may be the source of the obstruction by pushing the soft palate backwards. The limited impact of the soft palate is further confirmed by the 40-50% efficacy of procedures that remove the soft palate for treatment of OSA. However, it remains difficult to determine the specific impact of the tongue or any other tissues on a particular patient.

When a patient is suspected to have OSA due to significant daytime somnolence or excessive snoring with cessation of breathing as noted by the spouse, the patient is referred by his or her primary care physician to a sleep specialist. Sleep specialists are usually pulmonologists, psychiatrists, neurologists, or otolaryngologists that have a special interest in sleep disturbances. After clinical evaluation, the patient may be referred for a polysomnography, a study performed by sleep technicians and aimed to identify the presence and severity of OSA. The patient spends a night at a sleep study center where they quantify the number of episodes of obstruction that the patient has each hour (as mainly measured by airflow determinations) and how they relate to episodes of awakening (as measured with an electroencephalogram). Other physiological measurements performed include electrocardiography, pulse oximetry, electro-oculography, and abdominal pressure determinations.

In current practice, patients may be initially treated with non-invasive approaches such as Continuous Positive Airway Pressure (CPAP) and oral appliances. These methods require the patient to be followed closely with multiple visits to determine the ideal settings and compliance. If conservative methods are not adequate or if the patient is non-compliant, consideration is made for surgical strategies performed by otolaryngologists and oral and maxillofacial surgeons. These surgical procedures include uvulopalatopharyngoplasty (removal of tonsils, adenoids, uvula, and the posterior portion of the palate), genioglossus advancement (pulling of the tongue forward by pulling anteriorly a segment of the mandibular bone), and maxillomandibular reconstruction (cutting the maxillary and mandibular bones, advancing them, and securing them with screws and plates). Some newer technologies include the application of radiofrequency energy to the tongue in order to reduce its volume, the insertion of a polymer into the soft palate to increase its stiffness, and suturing the tongue to a screw placed in the mandible to advance the tongue.

The current strategies to treat OSA are ineffective, uncomfortable (leading to poor compliance), or significantly invasive. Improved methods of treatment for this condition are of great interest.

### PUBLICATIONS

U.S. Pat. Nos. 6,962,605; 6,800,090; 6,955,172; 5,792,067; 6,587,725; 6,502,574; 6,240,316; 6,578,580; 6,431,174; 6,523,541; 5,988,171; 6,450,169; 6,439,238; 4,198,967; 4,304,227; 6,413,254; 6,408,851; 5,649,540; 7,090,672. U.S. Patent application publications US2005/0092332; US2004/0149290; US2005/0191248; US2004/0139975; US2005/0092334; US2005/0115572; US2004/0112390; US2005/0121039; US2005/01251255; US2005/0199248 and US2006/0201519.

### SUMMARY OF THE INVENTION

Devices and methods are provided for the treatment of obstructive sleep apnea through a reversible and minimally invasive method that prevent obstruction by the tongue upon loss of muscle tone during sleep, while allowing for normal speech and swallowing. In the methods of the invention, an implantable device is inserted into the tongue to prevent obstruction of the airway. The implantable device is a flexible elongated structure conformed in a way that, once implanted, will prevent the tongue from rotating on its axis and cause obstruction. In some embodiments, the implantable device is a curved or jointed filament formed of a shape memory plastic or metal; of a sufficient length and diameter to provide support, when implanted. The mechanical properties are sufficient to restrain obstructive movement of the tongue during sleep while allowing for normal speech and swallowing during waking hours. The device may be implanted without fixation or anchoring to tissues, including hard tissues such as bone, etc. Once implanted, the device supports the tongue without reshaping or tensioning.

In some embodiments of the invention, a method of reducing sleep apnea is provided, the method comprising: implanting a device within the tongue of said patient, wherein the device restrains the passive movement of the soft tissue of the airway and prevents the collapse of the soft tissue into the airway.

In other embodiments of the invention, an implantation system is provided, wherein the system comprises an implantable device as set forth herein to restrain the passive movement of the tongue and prevent the collapse of the soft tissue into the airway, a catheter element adapted to guide the implantable device into the soft tissue of the tongue, and a trigger element adapted to release the device from the catheter element into the tongue. A removal system may also be provided, wherein the system comprises a catheter element which can be inserted into the tissue where the tongue supportive device is implanted. Located at the distal end of the removal system is a grasping member, and a manual means for actuating the grasping member is located at the proximal end of the device. The grasping member is used to grasp the tongue supportive device. In some embodiments of the subject devices, the grasping member is capable of being retracted into and protruded from a protective housing or sheath located at the distal end of the elongate, e.g. tubular device.

### BRIEF DESCRIPTION OF THE FIGURES

FIGS. 1A-1B are schematic drawings showing the anatomy of the tongue region. FIG. 1A is a sagittal-section; FIG. 1B is a cross section.

FIG. 2 is a schematic drawing of one embodiment of the invention, where the tongue supportive device is a linear, sigmoid structure.

FIG. 3 is a schematic drawing of one embodiment of the invention, where the tongue supportive device is a linear structure having a central angle and curved regions at the distal and proximal ends.

FIGS. 4A-4B are schematic drawings of one embodiment of the invention, where the tongue supportive device is a linear, curved structure. A single implant is depicted in FIG. 4A, and multiple implants in FIG. 4B.

FIG. 5 is a schematic drawing of one embodiment of the invention, where the tongue supportive device has a fishhook structure, having a crossbar at the distal region, and a curved region at the proximal end. Shown in FIG. 5, the crossbar is implanted in the root region of the tongue.

FIGS. 6A-6C are schematic drawings of one embodiment of the invention, where the tongue supportive device is a linear structure having a plurality of arms at the posterior end of the device. A lateral view of the device is shown in FIG. 6A; a top view in FIG. 6B; and an alternative arm configuration in FIG. 6C.

FIGS. 7A-7E illustrate some of the parameters of the tongue supportive device.

FIG. 8 is a schematic drawing of one embodiment of the invention, where the tongue supportive device is a linear structure having a plurality of arms at the posterior end of the device.

FIGS. 9A-9B are schematic drawings of one embodiment of the invention, where the tongue supportive device is a linear structure having a plurality of barbs along the length of the structure. Depicted in FIG. 9A is a straight implant; depicted in FIG. 9B is a sigmoid implant.

FIG. 10 is a schematic drawing of one embodiment of the invention, where the tongue supportive device is a Y-shaped structure having a two arms at the posterior end of the device.

FIGS. 11A and 11B are schematics of an implantation system (11A) and a withdrawal system (11B).

### DESCRIPTION OF THE PREFERRED EMBODIMENTS

Devices and methods are provided for the treatment of obstructive sleep apnea. In the methods of the invention, an implantable device is inserted into the tongue to prevent obstruction of the airway. The device is implanted such that the anterior end is positioned in the root of the tongue, and the posterior end lies in the base of the tongue or towards the tip of the tongue, without attachment to a hard tissue. The implantable device is a flexible elongated structure, which may be a straight, curved or jointed filament; of a sufficient length and diameter to provide support, when implanted, that is sufficient to restrain obstructive movement of the tongue during sleep.

The following description of preferred embodiments of the invention is not intended to limit the invention to these embodiments, but rather to enable any person skilled in the art to make and use this invention.

As used herein, the term “obstructive sleep apnea” in a human patient refers to episodes of partial and/or complete closure of the upper airway during sleep leading to breathing cessation, usually defined as greater than 10 sec. Symptoms include restlessness, snoring, recurrent awakening, morning headache, and excessive daytime sleepiness. Diagnosis is based on sleep history, physical examination, and polysomnography. In at-risk patients, sleep destabilizes the upper airway, causing partial or complete obstruction of the nasopharynx, oropharynx, or both. When breathing is diminished but not absent, the condition is called obstructive sleep hypopnea.

Anatomic risk factors include obesity (body mass index>30); an oropharynx “crowded” by a short or retracted mandible and prominent tongue, tonsils, lateral pharyngeal walls, or lateral parapharyngeal fat pad. The tongue has been implicated in at least 65% of cases. The limited success of the uvulopalatopharyngoplasty procedure is likely due to the tongue being the organ that is pushing back on the uvula, causing the obstruction. If the uvula is thus eliminated, the tongue will continue to contribute to the obstruction. Family history of sleep apnea is present in 25 to 40% of cases. OSA is also often found in association with chronic disease, such as hypertension, stroke, diabetes, gastroesophageal reflux disease, nocturnal angina, heart failure, and hypothyroidism.

Airway obstruction causes paroxysms of inspiratory effort, reductions in gas exchange, disruption of normal sleep architecture, and partial or complete arousals from sleep. Hypoxia and/or hypercapnia and sleep fragmentation interact to produce characteristic symptoms and signs.

A preliminary diagnosis based on identifiable risk factors and/or symptoms may be confirmed with polysomnography, which comprises continuous measures of breathing effort by plethysmography; airflow at the nose and mouth by flow sensors; O₂ saturation by oximetry; sleep architecture by electroencephalography (EEG) (for sleep stages), chin electromyography (for hypotonia), and electro-oculograms for rapid eye movements. Electrocardiography (ECG) is useful for determining whether arrhythmias occur with apneic episodes.

A common summary measure used to describe respiratory disturbances during sleep is the apnea-hypopnea index (AHI)—the total number of episodes of apnea and hypopnea as defined above during sleep divided by the hours of sleep time. AHI values can be computed for different sleep stages. The respiratory disturbance index (RDI) is a similar measure, which refers to the number of times per hour that blood O₂ saturation falls >3%. With an EEG, an arousal index (Al) can be computed, which is the number of arousals per hour of sleep. The Al may be correlated with AHI or RDI, but about 20% of apneas and desaturation episodes are not accompanied by arousals, or other causes of arousals are present. An AHI >5 is required for the diagnosis of OSA; values >15 and >30 indicate moderate and severe levels of sleep apnea, respectively. Snoring confers a 7-fold increase in the likelihood of having AHI >5. Adjunctive testing may include upper airway imaging, thyroid-stimulating hormone, and other tests as appropriate to assess chronic medical conditions associated with OSA.

The aim of treatment specific to OSA is to reduce episodes of hypoxia and sleep fragmentation; treatment is tailored to the patient and to the degree of impairment. Cure is defined as a resolution of symptoms with AHI reduction below a threshold, usually 10/h, or a 50% reduction in AHI. For the purposes of the present invention, a patient may be considered to be treated when OSA episodes, which may be measured by any one of the methods known in the art, e.g. AHI, RDI, Al, etc. are reduced by at least about 25%, at least about 50%, at least about 75%, at least about 85%, at least about 90%, at least about 95% or more.

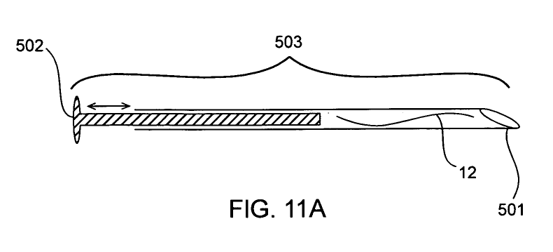

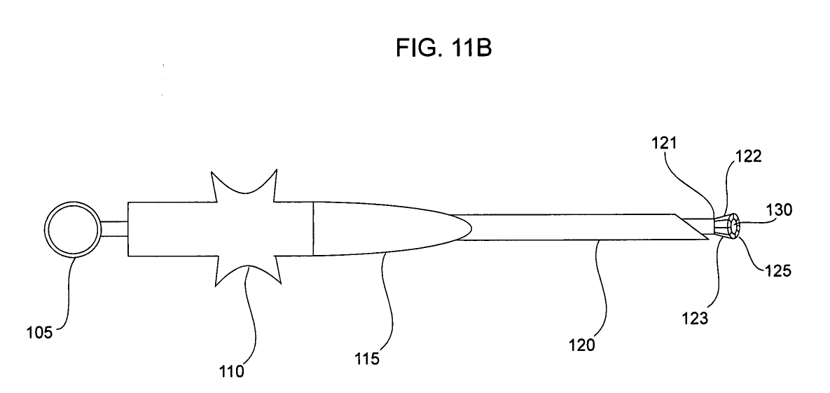

As shown in FIG. 11A, the implantation system 503 for the restraining element 12 of the invention (which also be referred to herein as an implantable device, or supportive element or device) includes a needlelike catheter element 501 having a sharp, open end suitable for puncture of soft tissues, including the tongue. The catheter is adapted to guide the restraining element 12 into the soft tissue, and a pushing element 502 is included to drive or push the restraining element 12 from the catheter element 501 into the soft tissue. For example, the restraining element is inserted into the catheter in a straightened configuration. The needle-like end of the catheter is used to puncture the soft tissue of the tongue, and is then driven or pushed into the tongue tissue until the needle end reaches the point at which the posterior end of the device will be positioned. The pushing element is then used to guide, or push the posterior end of the device into the tissue. In some embodiments, the curvature of the device serves to support the device in the tissue once a small portion has been inserted. The catheter is then withdrawn from the tissue, leaving the implanted device.

The device may be implanted in a way that its anterior end is less than about 1 cm, less than about 2.5 cm, less than about 5 cm, and not more than about 6 cm posterior to the mandible of the patient. This anterior end is located in the root of the tongue, which comprises the least mobile and most inferior part of the tongue, which extends from the posterior aspect of the mandible to the hyoid bone. The posterior aspect of the device is located in the base of the tongue, which comprises the posterior aspect of the tongue extending above the area of the epiglottis. This posterior end of the device is located at least about 0.25 cm, at least about 0.5 cm, at least about 0.75 cm, at least about 1 cm, and not more than about 2 cm deep into the tissue of the tongue. Due to the curvature of the device, it may bend anteriorly into the body or the tip of the tongue, which comprise the bulk and most mobile portion of the tongue, extending anteriorly from the base and superiorly from the root.

As shown in FIG. 11A, the catheter element 501 functions to make a small puncture site and guide the restraining element 12 into the tongue. In a first variation, the catheter element 501 is preferably a hollow tube adapted to hold the restraining element 12 in a straight configuration until the restraining element 12 is delivered into the tongue. The restraining element is preferably positioned and inserted behind the lower aspect of the chin, but may alternatively be positioned at the tip of the tongue through the oral cavity or any other suitable insertion position including the sides of the tongue. The catheter element 501 preferably guides the restraining element 12 into the tongue but may alternatively be inserted and guide the restraining element 12 into any suitable soft tissue of the patient's airway, such as the soft palate or lateral walls.

At the proximal end of the catheter, there is optionally included a manual means for pushing, or triggering insertion of the restraining element. The manual means may comprise a movable element for pushing or triggering, and a grip for improved control. The manual actuation means may be present in a variety of different configurations, so long as it is capable of providing for the requisite manual control.

A removal system is shown in FIG. 11B. At the distal end of a needle like catheter element 120 is a grasping device 125. The grasping member typically comprises a plurality of jaw elements 122, 123 that can be manipulated to grasp the implant, where the jaws may be arranged in a radial manner as shown in FIG. 11b. The jaw elements may be hinged 121 to allow for such movement. In some embodiments of the invention, the jaws will comprise a grasping surface, including a rubber, soft tacky adhesive, serrated surface, etc. 130 for improved grip on the implant. In certain embodiments, the device further comprises a jaw element locking means, which serves to lock the jaws in a given position, e.g. in a gripped position. At the proximal end of the elongate member 120 is a manual means 115 for actuating the grasping member. The manual means may comprise a movable element 105 for opening and closing the grasping member jaws, and a grip 110 for improved control. The manual actuation means may be present in a variety of different configurations, so long as it is capable of providing for the requisite manual control over the movement of the grasping members during use of the device. As such, any manual actuation means that can be operated by hand from a site external to the body and achieve the desired internal object manipulation or movement via the internal articulated member(s) during use may be present on the device. The manual actuation means typically includes one or more elements shaped or configured to be operated by fingers and/or a thumb which are operationally connected to the articulated members via wires, strings, cables, or other tensile elements, etc., to provide for the desired articulate member movement. Manual actuation means of interest include adaptations of those described in U.S. Patents of interest include: U.S. Pat. Nos. 5,997,567; 5,976,122; 5,891,162; 5,820,009; 5,797,959; 5,728,121; 5,713,919; 5,613,973; 5,549,636; 5,417,684; and 5,383,895; the disclosures of which are herein incorporated by reference.

The tongue supportive element 12 of the preferred embodiments functions to restrain the passive movement of the soft tissue structures of the airway and prevent the collapse of the soft tissue into the airway. The supportive element 12 is adapted to allow the normal movement and function of the tongue and other soft tissues, but will prevent the passive movement of the tissue due to gravity and negative inspiratory pressure of the airway, upon loss of tone during sleep. The restraining element 12 is preferably made of a biocompatible material that is sufficiently rigid to restrain the tissue from moving due to passive forces, such as gravity and pressure changes acting on the mass of the tissue, but is sufficiently flexible to allow the tissue to overcome the restraining element 12 with the active forces generated during speaking and swallowing. The active forces will move or elastically deform the restraining element 12. On loss of tone the restraining element will tend to its initial shape set and prevent the tongue from falling into the airway.

The supportive element 12 may be adapted to be adjustable. Preferably this is accomplished by inserting multiple restraining elements 12 until the desired effect is reached. Alternatively, the restraining elements 12 may be adjustable by adjusting the stiffness of the material, adding springs or joints, changing the geometry, or by any other suitable means such that the operator may adjust the restraining element 12 to best suit the patient. The restraining element 12 is further adapted to be formable such that it can be delivered into the soft tissue in a straight configuration and then, upon delivery, return to the pre-formed geometry. The restraining element 12 is preferably inserted into the tongue of the patient but may alternatively be inserted into any suitable soft tissue of the patient's airway, such as the soft palate or lateral walls, to prevent the collapse of the soft tissue into the airway and prevent obstruction of the airway.

### Configuration of the Device

The implantable device of the present invention is an elongated structure that provides support to the tongue. The device is conformed in a way that prevents the base of the tongue from rotating or elongating on its axis, therefore maintaining the cross-sectional area of the airway and preventing obstruction upon loss of muscle tone during sleep. By virtue of its mechanical and material properties, the device prevents the tongue from falling backwards into the airway but allows the tongue to move as needed for normal function such as speech and swallowing. Based on the average weight of the tongue, it has been estimated that at least about 0.25 N, at least about 0.5 N, at least about 0.75 N, at least about 1.0 N of force would be sufficient to maintain an open airway upon loss of muscle tone during sleep.

The Figures provided herein demonstrate a variety of configurations for the implantable device of the invention. In some embodiments, the tongue supportive device is a filamentous linear or branched structure, which may be curved, e.g. in a sigmoid or fishhook configuration, or straight. The structure may be smooth or barbed.

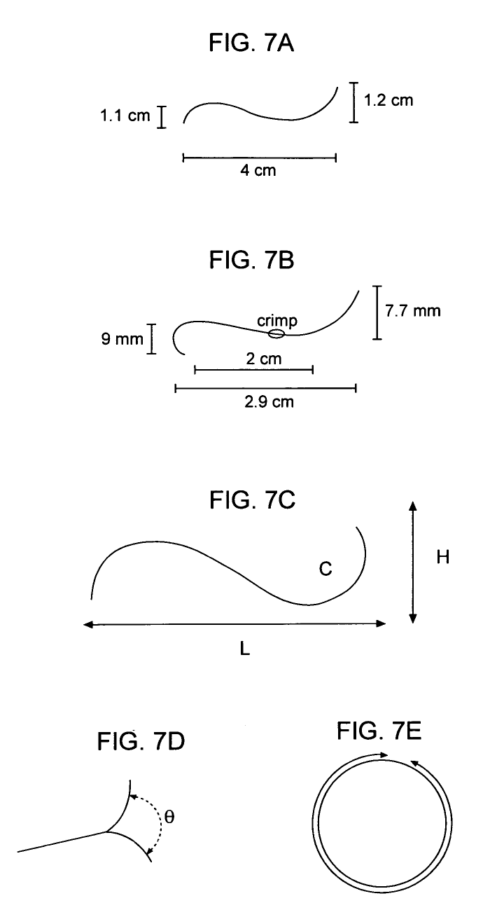

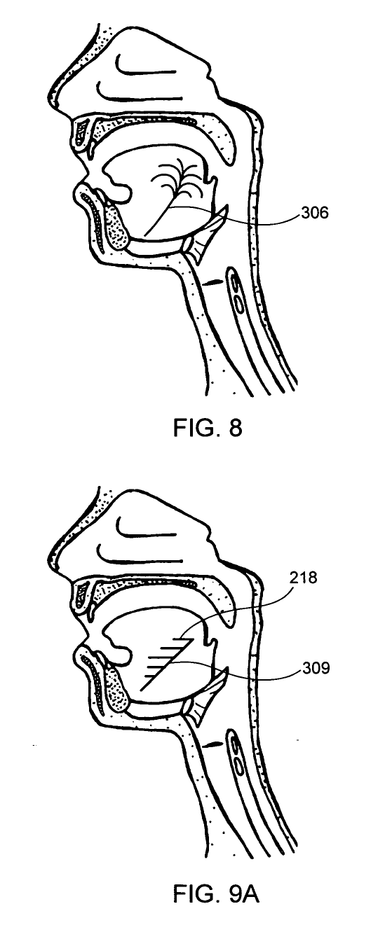

As shown in FIGS. 7A-7C, the effective length of the device, L, is usually at least about 1 cm, at least about 3 cm, at least about 4.5 cm, at least about 5 cm and not more than about 7.5 cm, usually not more than about 5 cm. The effective height of the device, H, is usually at least about 0.25 cm, at least about 0.5 cm, at least about 1 cm, and not more than about 2.5 cm. The configuration of the curves is such that one end serves as a support point for the device and the other allows the restraining of the tongue.

As described below and as shown in the Figures, a variety of configurations are provided for the implantable device. The following are exemplary.

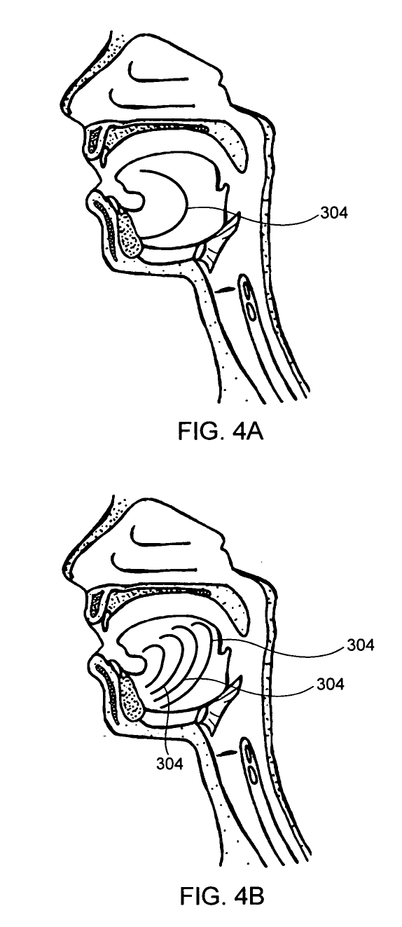

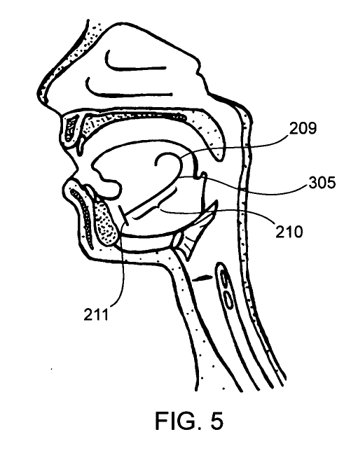

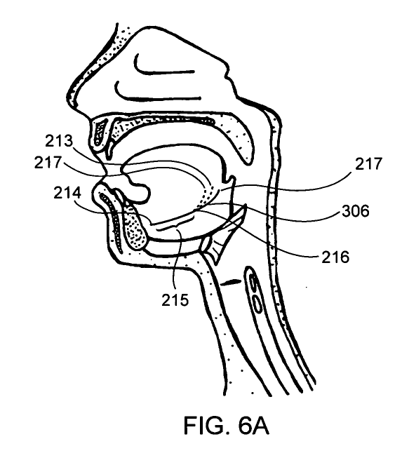

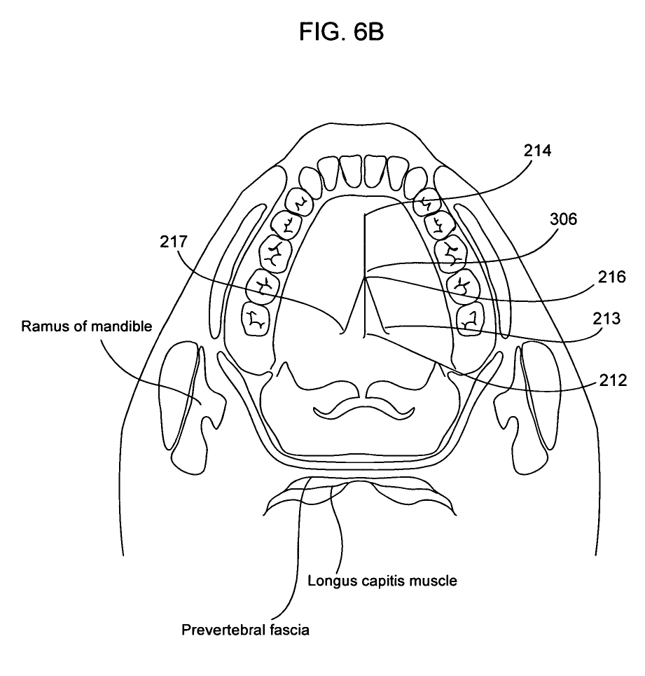

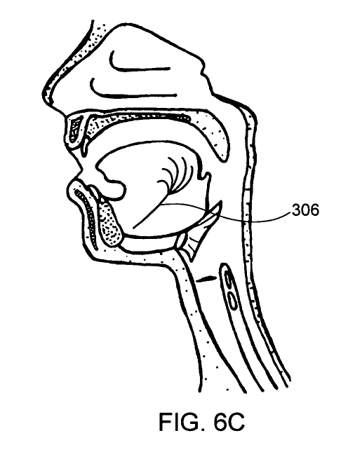

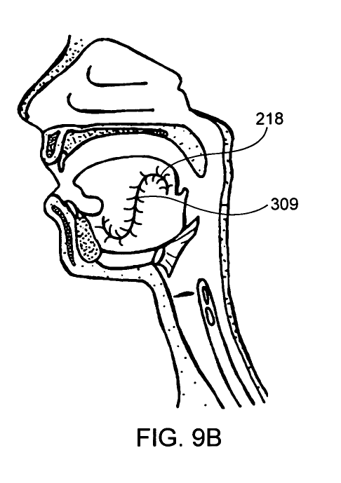

In some embodiment, the shape of the device is a single curve, e.g. a “fishhook” shape, which may have a straight segment, as shown in FIGS. 5A-5B, or be curved over the body of the device, as shown in FIGS. 4A-4B. The device may have a crossbar at one end, as shown in FIGS. 4A-4B. The device may have a sigmoid shape, e.g. as shown in FIG. 2, or in FIG. 9B. The sigmoid curve shape may have a straight internal segment, or be curved over the body of the device. A sigmoid device may optionally comprise one or more crossbar structures. The device may have a branched configuration, as shown in FIGS. 6A-6C, where the branched arms are angled such as to allow support of the tongue during sleep.

In embodiments where the device has one or more curved segments, the curve is an arcuate shape which may be of a constant radius, e.g. a radius of at least about 0.1 mm, at least about 0.25 mm, at least about 0.5 mm, not more than about 2.5 mm, not more than about 1 mm; or may have a variable radius, e.g. ranging from at least about 0.1 mm to not more than about 1 mm radius. The arcuate shape, A, will be at least about 10% of a circle, at least about 25%, at least about 50%, at least about 75%, and not more than about 90%, not more than about 80%.

In some embodiments of the invention, for example as shown in FIGS. 6A-6C and FIG. 8, the device comprises two or more arms, and may comprise three, four, five or more arms. Optionally, one or more, and in some embodiments all of the arms will have a curved shape, where the curves may be curved in the same or opposite direction, usually in the same direction. The angle between the arms, θ, may be from at least about 5 degrees, at least about 10 degrees, at least about 20 degrees, at least about 30 degrees, at least about 45 degrees, at least about 60 degrees, usually not more than about 180 degrees, not more than about 120 degrees.

Each terminus of the filament or ribbon may be flat, curved, or beaded. Where a bead is provided, it will usually have a diameter of not more than about twice the diameter of the filament or ribbon. For example, a filament of 0.33 mm diameter may accommodate a bead of from about 0.33 to about 0.66 mm diameter. The bead may be of the same material as the filament or ribbon, or may be of any biocompatible material that provides for a secure adhesion to the device.

A variety of biocompatible materials, known by those of skill in the art may be used to produce the implantable device of the invention. Biologically compatible metals include stainless steel, titanium, tantalum, gold, platinum, copper and the like, as well as alloys of these metals. Non-metallic biologically compatible materials include, without limitation, polymeric materials such as nylon, polyimide, polyamides, polyethylene or combinations thereof. In one embodiment of the invention, the implantable device is formed from a nitinol alloy. Nitinol is a family of intermetallic materials, which contain a nearly equal mixture of nickel (55 wt. %) and titanium. The equiatomic composition forms the basis of many nitinol alloys. Adding an additional nickel up to an extra 1% is the most common modification. This increases the yield strength of the austenitic phase while at the same time depressing the transformation temperature. Other common additions are made to alter the phase transformation temperature, such as iron and chromium which lower the temperature. Copper can also be added to lower the stress required to deform the martensitic phase and decreases hysteresis.

In some embodiments of the invention, the device is formed of a shape memory metal, e.g. nitinol. Nitinol has properties of super-elasticity and shape memory. The device may initially be shape set into a desired geometry, as explained in detail below. At delivery the device is deformed to have a straight, or tensed, configuration. Upon release from the delivery system, the shape memory of the material will provide forces that tend to restore the initial geometry, thus exerting supportive forces on the tongue. These forces will restrain the tongue from collapsing into the airway upon loss of tone. The super elasticity of the material allows normal speech and swallowing of the tongue after implantation of the device.

The entire implantable device may be formed from a single filament, or wire, if desired, or joints and arms may be separately formed and attached by any suitable method, as known in the art, e.g. glued, welded, crimped, etc. A crimp may be of a square or rounded geometry. Filaments may have any cross-sectional geometry, e.g. square, round, oval, triangular, ribbon, etc. Where applicable, the arms may be made of the same or different material, and combinations of materials may be used. Arms may also be variable in length, or may be of equal lengths.

The diameter of the filament or formed implantable device can vary. Generally a filament will be at least about 0.1 mm, at least about 0.25 mm, at least about 0.33 mm, at least about 0.5 mm, and not more than about 2 mm, usually not more than about 1 mm, and may be around about 0.33 mm diameter. The cross-section of a filament need not be constant along its entire length, but may include portions having a larger or smaller cross-section as desired. Where the device is formed of a ribbon filament, the ribbon width will usually be about 0.25 mm, at least about 0.5 mm, at least about 0.75 mm, at least about 1.0 mm and not more than about 2 mm. The thickness of the ribbon filament will usually be at least about 0.1 mm, at least about 0.25 mm, at least about 0.5 mm, at least about 0.75 mm, but not more than about 1 mm.

### Methods of Use

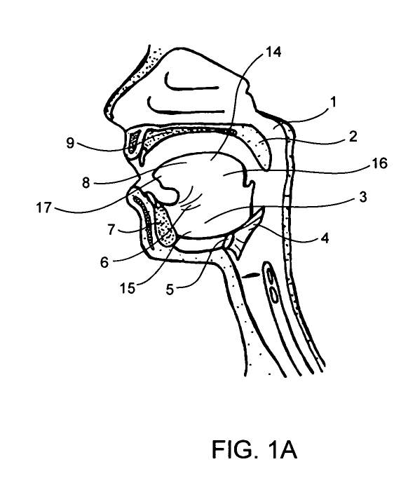

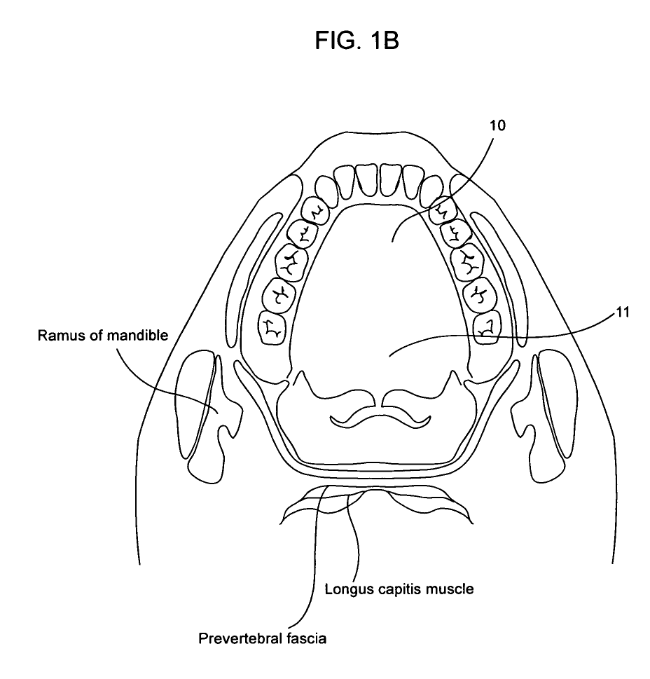

In the methods of the invention, the implantable device may be inserted through any suitable means, including through the soft tissue of the lower jaw; or coming from above the tongue, or from its sides. Implantation may also be performed by directly placing the device through an open surgical procedure. Usually a catheter device will be used to guide the implant into position, and the implantation may be performed under local or general anesthesia. When the implant is in position, the anterior portion of the device is situated in the less mobile portion of the tongue, which comprises the relatively fixed root of the tongue as shown in FIGS. 1A-1B, and extends up to the mandible bone. FIG. 1A depicts the ventral 15 and dorsal 14 regions of the oral region when shown in sagittal-section, with the root 3, epiglottis 4, hyoid bone 5, genioglossus muscle 6, mandible 7, and hard palate 9. The body of the tongue 8 including the tip 17 is mobile. The root 3 is immobile, and the base 16 is relatively mobile compared to the root. A top view of the tongue region, shown in FIG. 1B, illustrates the anterior 10 and posterior 11 positions.

In some embodiments of the invention, the device is not fixed or attached to the bone or other hard tissue, although it may be implanted in close proximity. The posterior portion of the device is situated in the region of the tongue close to its base. It may curve at the base of the tongue and extend into different areas including the body and tip of the tongue.

To remove the implant, if needed, a second catheter device is inserted into the tongue. The implant is grasped with the jaw of the grasping element. The grasping element holding the implant is withdrawn into the catheter, and removed it from the tissue through the sheath of the removal device.

By advancing the tongue, the devices of the invention offer patients a reliable, simple, minimally-invasive, reversible, and adjustable way to treat varying severities of OSA. The device can serve as a standalone treatment for patients with mild and moderate degrees of OSA in which the tongue is the major contributor to the disease. Optionally, the device is combined with other strategies that tackle the soft palate and lateral walls of the pharynx in patients with severe disease and in those in which the tongue is not the only contributor. Due to the simplicity and reversibility of the methods of the invention, the implants can be used as first step in the treatment of a wide range of OSA patients. Other treatment strategies aimed towards reducing obstruction from other areas of the airway may be added if needed to complement the efficacy. By reducing obstruction of the airway, the long-term consequences of OSA, including cardiovascular complications and premature death, may be addressed.

Turning to the drawings, the implantable device may be one of several variations. In one embodiment, as shown in FIG. 2, the supportive element 302 is configured into a curved sigmoid geometry at ends 201 and 202, which allows the implant to be supported by the genioglossus muscle at a connection point near the lower jaw bone and at a point in the superior-posterior aspect of the tongue. The device is implanted such that the anterior end 202 is positioned in the root of the tongue, and the posterior end 201 which lies in the base of the tongue. As shown in the figure, the device may be implanted such that the anterior end 202 curves downward and the posterior end 201 curves upward. The element 12 prevents the soft tissue from collapsing into the airway by restraining the passive movement of the genioglossus muscle within the predetermined length of the restraining element 12.

In one embodiment, the supportive device 302 is a sigmoid nitinol filament with a length of from 1 to 4 cm, and a diameter of 0.2 to 0.4 mm, having a curved end 202 where the curve has a radius of from 0.25 to 1 cm, and the arc is at least a quarter circle, 0.25%, to three-quarters of a circle, 0.75%, and is usually an arc of from 40% to 60% of a circle. The center region of the device 203 extends from about 0.5 to about 2 cm and maybe straight or a continuous curve with the ends. The curved end 201 has a radius of from 0.25 to 1 cm, and the arc is at least a quarter circle, 0.25%, to three-quarters of a circle, 0.75%, and is usually an arc of from 40% to 60% of a circle.

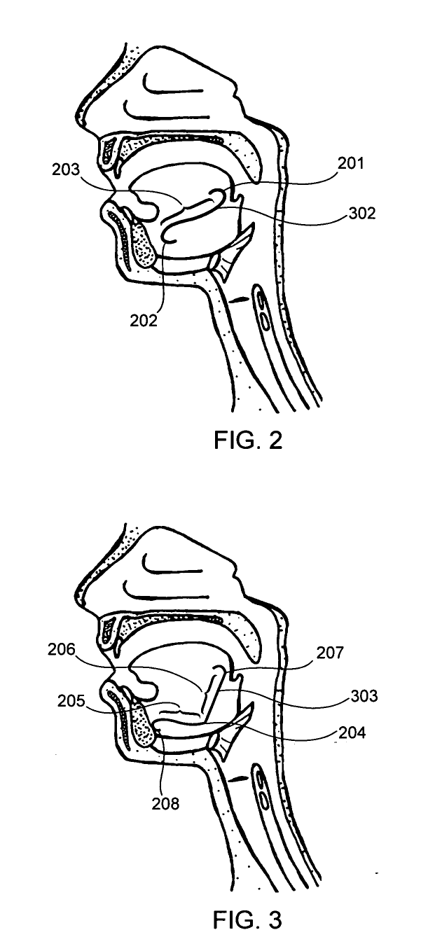

An alternative embodiment is shown in FIG. 3, where the restraining element 303 is a linear shape with a central angle 204 of from about 60° to about 120°. The ends 207 and 208 are curved, where the curves may be in the same or different directions. The curve 207 has a radius of from 0.25 to 1 cm, and the arc is at least a quarter circle, 0.25%, to three-quarters of a circle, 0.75%, and is usually an arc of from 40% to 60% of a circle. The center region 206 extends from about 0.1 to about 0.25 cm and may be straight or form a continuous curve with the end 207. The curved end 208 has a radius of from 0.25 to 1 cm, and the arc is at least a quarter circle, 0.25%, to three-quarters of a circle, 0.75%, and is usually an arc of from 40% to 60% of a circle. The center region 205 extends from about 0.1 to about 0.25 cm and may be straight or form a continuous curve with the end 208.

An alternative embodiment is shown in FIGS. 4A and 4B. The supportive element 304 is a single curve or parabolic shape, having a path length of from 1 cm to about 7.5 cm, with a curve of radius 1 to 3 cm. The implant may be positioned such that the open end of the curve or parabola faces in the dorsal position, as shown in FIG. 4A. As shown in FIG. 4B, a plurality of implants 304 may be positioned parallel to each other, where the radius of curves may be the same or different. For example, the dorsal implant may be a smaller curve radius relative to the ventral implant. The implants may be positioned from about 0.1 to 1 cm from each other.

Shown in FIG. 5A, the supportive element 305 is a fishhook shape, i.e. a curved geometry with a base, or crossbar. The crossbar 211 is roughly perpendicular to the central region 210, and is from about 0.1 cm to about 0.5 cm in length, typically connected to the central region at the midpoint. The curved end 209 has a radius of from 0.25 to 1 cm, and the arc is at least a quarter circle, 0.25%, to three-quarters of a circle, 0.75%, and is usually an arc of from 40% to 60% of a circle. The center region 210 extends from about 0.25 to about 2.5 cm and may be straight or form a continuous curve with the end 209. When implanted, the crossbar 211 is positioned in the root of the tongue, near the connection point of the tongue to the lower jawbone and the curved portion 209 is positioned at the superior-posterior aspect of the tongue, with a dorsal open end of the curve.

Shown in FIGS. 6A to 6C, the supportive element 306 is configured into a pronged geometry, which has an end 214, a center region 215 a junction point 216, which may be a crimp, weld, etc., and two or more arms 212, 213, 217, for example having two, three, four or five arms. The end 214 may be straight or curved. When curved, the curve has a radius of from 0.25 to 1 cm, and the arc is at least a quarter circle, 0.25%, to three-quarters of a circle, 0.75%, and is usually an arc of from 40% to 60% of a circle. The junction 216 lies between the anterior and posterior ends, where the length ratio of anterior to posterior segments is from around about 1:1, 1:2; 1:3 to 1:4. The angle between each arm is as previously described, where usually the total spread about the outward arms is not more about 120°, not more than about 90° and is at least about 30°, at least about 45°, at least about 60° or more. The angle may be constant, or variable for example where the arms curve away from each other. The posterior region 215 is usually from about 0.5 cm in length to about 1.5 cm in length, while the arms range from about 0.25 to 0.5 cm in length. Each arm, 212, 213, 217, etc. is independently straight or curved, and where curved, each arm may be curved in the same or opposite direction. When curved, the curve has a radius of from 0.25 to 1 cm, and the arc is at least a quarter circle, 0.25%, to three-quarters of a circle, 0.75%, and is usually an arc of from 40% to 60% of a circle.

In one embodiment, the device has three or four arms, and is formed of nitinol wire having a diameter from 0.2 to 0.4 mm in diameter, where the arms are attached by a crimp at the junction point 216. Each arm is curved in the same direction in the superior-inferior plane, while the end 214 is straight.

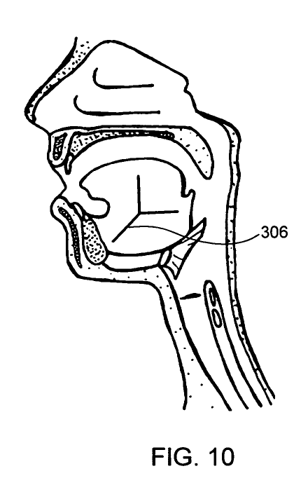

An alternative embodiment of 306, shown in FIG. 8, has a plurality of arms of at least five and not more than about ten arms. The embodiment shown in FIG. 10 features straight arms and end, having two arms.

In the embodiments shown in FIGS. 9A and 9B, the restraining element 309 has a geometry as shown in any of the previous embodiments, e.g. in FIG. 2, where the main filament of the device comprises multiple protrusions or barbs 218 extending from the element at various points along the element. The protrusions consist of flexible material, for example a shape memory material, etc., and may be of the same material as the body of the device, or a different material. The protrusions may be provided in a single plane, or may radiate from the bosy of the device in multiple planes, e.g. in a radial fashion. The protrusions will be from about 0.05 cm, about 0.1 cm, about 0.2 cm, to about 1 cm in length. The density of protrusions over the body of the device will usually be at least about 1 protrusion per 5 cm; at least about 1 protrusion per 2.5 cm, at least about 1 protrusion per 1 cm, at least about 1 protrusion per 0.5 cm, at least about 1 protrusion per 0.1 cm

Although omitted for conciseness, the preferred embodiments include every combination and permutation of the various supportive elements, implantation elements and withdrawal elements.

### Analysis of Forces

The methods of the present invention are not to be bound by any theory of action. However, in the assistance of design calculations for embodiments of interest, the forces acting on the tongue may be modeled using a simple approximation of the tongue and associated forces in the supine position. The tongue is a mobile organ composed of multiple muscles. It is fit into a mobile area superiorly and a fixed root inferiorly. The fixation points of the root of the tongue are the mandible anteriorly and the hyoid bone posterior and inferiorly.

For purposes of calculation, the tongue may be modeled as a cylinider shaped tissue secured at its root, and with a non-homogeneous density that contributes to the location of its center of gravity (C_{g}) towards its superior aspect (FIG. 2). The force of gravity (F_{g}) acts perpendicular to the mandible pulling the tissue in a downward direction. Normally, the muscular tone within the muscle would be sufficient to counteract this force. However, upon loss of tone, gravity dominates and a moment is formed around the pivot point (Pₚ) causing the tissue to rotate and collapse into the airway.

Pₚ: Pivot point

C_{g}: center of gravity

F_{g}: force due to gravity

F_{gx}: Force (as defined in the x-direction)

F_{gy}: Force (as defined in the y-direction)

θ: Offset angle from normal

Mₜ: Mass of the tongue

g: Acceleration due to gravity (9.8 m/s²

Eₜ: Elastic modulus of tissue

d: Diameter of implant

Fₜ: Force of the tongue

In the toned form, the forces (F) and torques (τ) in the static system must sum to zero. For the purposes of the estimation set forth herein, these calculations will only include the y-direction as this is the major contributor to occlusion in the simulated tongue.
Στ_{y}=0 and ΣF_{y}=0

The only forces acting on the tongue are the toning force (Ft) which is naturally present in the tongue and is supplemented by a device of the present invention. In this case, the balance is shown in equation 1, where the force due to gravity is defined by the average mass of a tongue multiplied by gravity acting in the defined direction depending on the rotation around the pivot point accounted for by the offset angle. These values are mirrored in the torque balance, assuming forces occur at the same points.
F_{gy}=−Fₜ=Mₜg sin θ (1)

Modeling the device of the present invention as a lever beam that has an intermediate load applied, the values from the tongue model can be extended to determine material dimensions and properties. Assuming an implant length of the device of the present invention of Lᵢ (cm) and an applied load occurring one third of the distance from the distal end of the device (a), equation 2 describes maximum displacement (Wₘₐₓ), the allowable bending that an implant would undergo.
Wₘₐₓ=(F_{gy}a²/(6 EI))(3L−a) (2)

where E is the elastic modulus of the implant and I is the moment of inertia (I=πd⁴/64). In this case, the moment of inertia for the device is defined as circular cross-section of diameter d (assuming that the device is a cylinder). Solving for the diameter of the implant needed for the force defined through equation 1 yields the following equation.
d=((F_{gy}a²64(3L−a)Wₘₐₓ6Eπ))^{1/4} (3)

It should be cautioned that these calculations were performed on a basic model of complex anatomy and assumed several variables. Based on equation 3 and using common material properties for shape memory alloys and approximate geometries, these calculations demonstrate that an implant can be designed to provide complete support to counteract gravity in the described system.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. The examples are put forth so as to provide those of ordinary skill in the art with a complete disclosure and description of how to make and use the subject invention, and are not intended to limit the scope of what is regarded as the invention.

What is claimed is:

1. A method of treating obstructive sleep apnea in a patient, the method comprising:
implanting a flexible filamentous device within the tongue of said patient, with said device anterior end positioned in the root of the tongue, said posterior end positioned in the base or tip of the tongue, without attachment to a hard tissue, and
wherein the shape memory of said device exerts forces that restrain passive movement of soft tissue of the airway of said patient.

2. The method according to claim 1, wherein episodes of obstructive sleep apnea in said patient are reduced by at least about 50%.

3. The method according to claim 1, wherein said device is formed of a shape memory material.

4. The method according to claim 3, wherein said implanting comprises:
inserting said flexible filamentous device into a catheter;
puncturing tissue of the tongue of said patient with said catheter;
driving said catheter into said tissue;
guiding said flexible filamentous device into said tissue; and
removing said catheter from said tissue.

5. The method according to claim 4, wherein prior to said implanting, said flexible filamentous device is shape set into a curved configuration; which configuration is straightened by said inserting into said catheter, and wherein upon release from said catheter, the shape memory of the material will provides forces that tend to restore the initial geometry, and exert supportive forces on the tongue.

6. The method according to claim 5, wherein said shape memory material is nitinol.

7. The method according to claim 6, wherein said flexible filamentous device is a filament of from 0.1 mm to 5 mm diameter.

8. The method according to claim 6, wherein said device is a filamentous ribbon of from 0.25 mm to 2 mm in width and 0.1 to 1 mm thickness.

9. The method according to claim 6, wherein said flexible filamentous device is from 2 to 10 cm in length.

10. The method according to claim 9, wherein said flexible filamentous device comprises at least one curved segment, wherein said segment is an arcuate shape of constant or variable radius from 0.1 mm to 1 mm.

11. The method according to claim 10, wherein said flexible filamentous device comprises two or more arms.

12. The method according to claim 10, wherein said method is reversible.

13. The method according to claim 10, wherein said flexible filamentous device is configured into a curved sigmoid geometry.

14. The method according to claim 13, wherein flexible filamentous device is a sigmoid nitinol filament with a length of from 1 to 4 cm, and a diameter of 0.2 to 0.4 mm, having a curved posterior end where the curve has a radius of from 0.25 to 1 cm, a center region extending from 0.5 to 2.5 cm, and a curved posterior end where the curve has a radius of from 0.25 to 1 cm.

15. The method according to claim 13, wherein said flexible filamentous device comprises a plurality of barbs extending from the element.

16. The method according to claim 10 wherein said flexible filamentous device is a single curve or parabolic shape, having a path length of from 1 cm to about 7.5 cm, with a curve of radius 1 to 3 cm.

17. The method according to claim 10 wherein said flexible filamentous device is a fishhook shape with a posterior curved end having a radius of from 0.25 to 1 cm and an anterior end comprising a crossbar of from 0.1 cm to 0.5 cm in length.

18. The method according to claim 10 wherein flexible filamentous device is configured into a pronged geometry, having an end, a center region, a junction point, and two or more arms.

19. The method according to claim 18, wherein the total spread between said arms is from 30 to 120o.

20. The method according to claim 19, wherein each of said arms is curved in a radius of from 0.25 to 1 cm.

21. The method according to claim 20, wherein each arm is curved in the same direction in the superior-inferior plane.

22. The method according to claim 21, wherein said device has three or four arms.

23. A flexible filamentous device suitable for use in the methods set forth in claims 1.

24. A system for treatment of obstructive sleep apnea in a patient, said system comprising a device as set forth in claim 23; and an insertion system comprising a needlelike catheter element, and a pushing element.

25. The system according to claim 24, further comprising a removal system comprising a needlelike catheter element, and a grasping device.
